(19) United States
(12) Patent Application Publication
(10) Pub. No.: US 2010/0210722 A1
(43) Pub. Date: Aug. 19, 2010

(54) COMPOSITION FOR PREVENTING OR TREATING LIPID METABOLIC DISORDERS COMPRISING FUCOXANTHIN OR MARINE PLANT EXTRACT CONTAINING SAME

(75) Inventors: Yong Chul Shin, Gyeongsangnam-Do (KR); Myung-Sook Choi, Daegu (KR); Myoung-Nam Woo, Gyeongsangnam-Do (KR); Kyung Hwa Jung, Gyeongsangnam-Do (KR); Ki Seok Kim, Gyeongsangnam-Do (KR)

Correspondence Address: EDWARDS ANGELL PALMER & DODGE LLP, P.O. BOX 55874, BOSTON, MA, 02205, US

(73) Assignee: AMICOGEN, INC., Jinju, Gyeongsangnam-Do (KR)

(21) Appl. No.: 12/682,493

(22) PCT Filed: Oct. 7, 2008
(86) PCT No.: PCT/KR08/05868

§ 371 Date: Apr. 9, 2010

(30) Foreign Application Priority Data
Oct. 10, 2007 (KR) 10-2007-0101968
Oct. 10, 2007 (KR) 10-2007-0101976

(51) Int. Cl. A61K 31/22; C07C 69/00; A61P 3/00

(52) U.S. Cl. 514/546; 560/129

(57) ABSTRACT

The present invention relates to a composition for the prevention or treatment lipid metabolic disorders comprising fucoxanthin or marine plant extract comtaining the same as an effective indredients. Fucoxanthin or a marine plant extract comprising the same is effective in reducing weight increase and reducing triglyceride and cholesterol level in liver tissue, or plasma through inhibiting the synthesis of fatty acid and promoting the oxidation of fatty acid. Therefore, the composition comprising fucoxanthin or a marine plant extract comprising the same as an effective ingredient may be effectively used for the prevention and treatment of lipid metabolic disorders.

### TECHNICAL FIELD

This application claims priority to Korean Patent Application No. 2007-0101968, filed on Oct. 10, 2007, and Korean Patent Application No. 2007-0101976, filed on Oct. 10, 2007, the contents of which are hereby incorporated by reference.

The present invention relates to a composition for the prevention or treatment lipid metabolic disorders comprising fucoxanthin or marine plant extract containing the same as an effective ingredient.

### BACKGROUND ART

Metabolic disorders include such diseases as obesity, diabetes, fatty liver, hyperlipidemia, arteriosclerosis, atherosclerosis, hypertension, stroke, myocardial infarction and the like. Typically, more than one of such conditions occurs simultaneously in a patient. The metabolic disorders are not caused by different reasons. Basically, they result from abnormal metabolism of sugars or lipids.

Especially, lipid metabolic disorders are caused by abnormal lipid metabolism. In particular, excessive accumulation of lipid leads to such diseases as obesity, diabetes, fatty liver, hyperlipidemia, arteriosclerosis, atherosclerosis, hypertension, stroke and myocardial infarction. Of the disease, hyperlipidemia is generally classified into hypercholesterolaemia in which total blood cholesterol level is high, hypertriglyceridemia in which triglyceride level is high, and a case in which both levels are high. The hyperlipidemia may induce and promote arteriosclerosis, and, in severe case, may lead to angina pectoris, myocardial infarction, or the like. The fatty liver retards the recovery of the liver due to increased load of liver detoxication, when the liver is damaged due to alcohol intake, drug addiction, etc. When left alone without treatment, it may develop into fatty hepatitis, fatty liver cirrhosis, liver cancer, etc. and may cause diabetes, hypertension, and the like.

Because of excessive fat consumption in the dietary life of the modern people, the number of lipid metabolic disorder patients is increasing rapidly. Therefore, development of a material capable of effectively preventing and treating the lipid metabolic disorders is highly required.

Fucoxanthin, which is a carotenoid with the following Chemical Formula 1, is mainly present in marine plants such as wakame, gulfweed, dashima, hijiki, and the like. It gives them a brown or olive-green color. Fucoxanthin is known to have anticancer [Das, S. K. et al., Biochim. Biophys. Acta., 2005, 1726(3):328-335], anti-inflammatory [Shiratori, K. et al., Exp Eye Res. 2005, 81(4):422-428] and anti-angiogenic [Sugawara, T. et al., J. Agric. Food Chem. 2006, 54(26):9805-9810]activities. However, there is no report about fucoxanthin's effect in preventing or treating lipid metabolic disorders as yet.

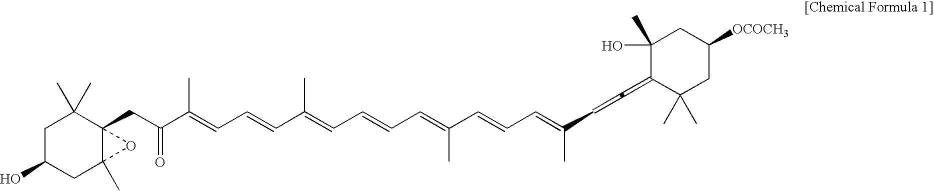

### DISCLOSURE

### Technical Problem

The inventors of the present invention have carried out researches on the treatment of lipid metabolic disorders. In doing so, they found out that fucoxanthin or a marine plant extract comprising the same is effective in inhibiting the synthesis of fatty acid and promoting oxidation of fatty acid, thereby inhibiting the generation of triglyceride and cholesterol.

Accordingly, an object of the present invention is to provide a pharmaceutical composition for preventing or treating lipid metabolic disorders comprising fucoxanthin or a marine plant extract comprising the same as an effective ingredient.

Another object of the present invention is to provide a food composition for preventing or treating lipid metabolic disorders comprising fucoxanthin or a marine plant extract comprising the same as an effective ingredient.

Another object of the present invention is to provide a feed composition comprising fucoxanthin or a marine plant extract comprising the same as an effective ingredient.

Another object of the present invention is to provide an use of fucoxanthin or a marine plant extract comprising the same for the preparation of a therapeutic agent for lipid metabolic disorders.

Another object of the present invention is to provide an use of fucoxanthin or a marine plant extract comprising the same for the preparation of a food composition.

Another object of the present invention is to provide an use of fucoxanthin or a marine plant extract comprising the same for the preparation of a feed composition.

Another object of the present invention is to provide a method for preventing or treating lipid metabolic disorders comprising administering fucoxanthin or a marine plant extract comprising the same to a subject in need thereof as an effective amount.

### Technical Solution

The present invention has been made to attain the aforesaid objects.

In an aspect, the present invention provides pharmaceutical composition for preventing or treating lipid metabolic disorders comprising fucoxanthin or a marine plant extract comprising the same as an effective ingredient.

In another aspect, the present invention provides a food composition for preventing or treating lipid metabolic disorders comprising fucoxanthin or a marine plant extract comprising the same as an effective ingredient.

In another aspect, the present invention provides a feed composition comprising fucoxanthin or a marine plant extract comprising the same as an effective ingredient.

In another aspect, the present invention provides an use of fucoxanthin or a marine plant extract comprising the same for the preparation of a therapeutic agent for lipid metabolic disorders.

In another aspect, the present invention provides an use of fucoxanthin or a marine plant extract comprising the same for the preparation of a food composition.

In another aspect, the present invention provides an use of fucoxanthin or a marine plant extract comprising the same for the preparation of a feed composition.

In another aspect, the present invention provides a method for preventing or treating lipid metabolic disorders comprising administering fucoxanthin or a marine plant extract comprising the same to a subject in need thereof as an effective amount.

Hereinafter, the present invention will be described in more detail.

As used herein, a “lipid metabolic disorder” refers to a disease caused by an abnormal lipid metabolism in the body, particularly by an excessive accumulation of lipids in the body. The “lipid metabolic disorder” may be selected from the group consisting of obesity, diabetes, fatty liver, hyperlipidemia, arteriosclerosis, atherosclerosis, hypertension, stroke and myocardial infarction, but is not limited thereto.

The pharmaceutical composition for the prevention or treatment of lipid metabolic disorders of the present invention comprises fucoxanthin or a marine plant extract comprising the same as an effective ingredient.

Fucoxanthin has a structure represented by the following Chemical Formula 1:

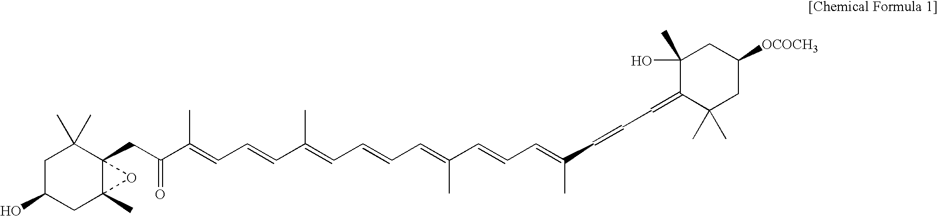

The marine plant extract comprising fucoxanthin may be obtained by a common extraction method, without special limitation. Preferably, it may be obtained by extracting marine plants with water, spirit, hexane, ethyl acetate, isopropyl alcohol, acetone or a mixture thereof at 10-50° C. for 1-48 hours. The marine plant may be any one as long as it contains fucoxanthin. Preferably, it may be at least one selected from the group consisting of wakame, dashima, gulfweed and hijiki, but is not limited thereto.

In an embodiment of the present invention, spirit and water were added to dry wakame powder and extraction was carried out for 6 hours to obtain a fucoxanthin extract (see Example 1). Thus obtained wakame extract was prepared into highly pure fucoxanthin by carrying out further extraction by adding spirit, hexane and acetone (see Example 2).

A test group (see Reference Example 1) which was fed with the fucoxanthin extract along with a high fat diet exhibited remarkably reduced body weight increase as compared to a control group (see Test Example 1) which was fed only with a high fat diet.

Further, the test group exhibited significantly reduced triglyceride and cholesterol levels in the liver tissue or plasma as compared to the control group. On the contrary, the test group exhibited significantly higher triglyceride and cholesterol levels in feces as compared to the control group. This indicates that the fucoxanthin extract inhibits the intake of cholesterol and triglyceride (see Test Example 2).

Further, in order to confirm the inhibition activity of the fucoxanthin extract against synthesis of fatty acid, change of the activity of fatty acid synthase (hereinafter, “FAS”), glucose-6-phosphate dehydrogenase (hereinafter, “G6PD”) and malic enzyme (hereinafter, “ME”), which are enzymes involved in the synthesis of fatty acid, was measured in the test group fed with the fucoxanthin extract. As a result, activity of FAS, ME and G6PD in the adipose tissue or liver tissue was lower in the test group fed with the fucoxanthin extract as compared to the control group (high fat diet group). This indicates that the fucoxanthin extract can inhibit the activity of enzymes involved in the synthesis of fatty acid in the adipose tissue or liver tissue (see Test Example 3).

Further, in order to confirm the inhibition activity of the fucoxanthin extract against synthesis of triglyceride, change of the activity of phosphatidate phosphohydrolase (PAP), which converts fatty acid and 1,3-diglyceride into triglyceride via phosphatidic acid pathway, was measured in the test group fed with the fucoxanthin extract. As a result, decreased activity of PAP was observed in the test group fed with the fucoxanthin extract. This indicates that the fucoxanthin extract can inhibit the activity of the enzyme involved in the synthesis of triglyceride (see Test Example 4).

Further, in order to confirm the activity of the fucoxanthin extract of promoting oxidation of fatty acid, change of the activity of carnitine palmitoyltransferase (CPT), which is involved in the oxidation of fatty acid, and change of β-oxidation activity were measured. As a result, the test group fed with the fucoxanthin extract exhibited significantly increased CPT activity and β-oxidation activity as compared to the control group fed only with the high fat diet. This indicates that the fucoxanthin extract can stimulate the activity of the enzyme involved in the oxidation of fatty acid (see Test Example 5).

Further, the effect of the fucoxanthin extract on the mRNA expression levels of fatty acid synthase and oxidase was confirmed in adipose tissue and liver tissue, respectively. As a result, the test group fed with the fucoxanthin extract exhibited' significantly increased the mRNA expression levels of CPT and β-oxidase, which are involved in the oxidation of fatty acid in the adipose tissue, as compared to the control group fed only with the high fat diet. In contrast, the mRNA expression levels of FAS, ME and G6PD, which are involved in the synthesis of fatty acid, significantly decreased as compared to the control group (see Test Example 6-1). In addition, the test group fed with the fucoxanthin extract exhibited significantly increased the mRNA expression level of peroxisome proliferator-activated receptor a (PPARα), which is involved in the oxidation of fatty acid in liver tissue, and the mRNA expression level of lipoprotein lipase (LP), which is an enzyme that hydrolyzes triglyceride, as compared to the control group fed only with the high fat diet. In contrast, the mRNA expression level of ME, which is involved in the synthesis of fatty acid, decreased significantly as compared to the control group (see Test Example 6-2).

To conclude, fucoxanthin or a marine plant extract comprising the same reduces the expression of mRNA for enzymes involved in the synthesis of fatty acid, thereby inhibiting the synthesis of fatty acid, and induces the expression of mRNA for enzymes involved in the oxidation of fatty acid, thereby promoting the oxidation of fatty acid. As a result, it reduces weight increase, and triglyceride and cholesterol level in liver tissue or plasma in spite of feeding with a high fat diet.

Accordingly, fucoxanthin or a marine plant extract comprising the same can be useful as an effective ingredient of a pharmaceutical composition for the prevention or treatment of lipid metabolic disorders.

Preferably, the lipid metabolic disorder is selected from the group consisting of obesity, diabetes, fatty liver, hyperlipidemia, arteriosclerosis, atherosclerosis, hypertension, stroke and myocardial infarction, but is not limited thereto.

The pharmaceutical composition according to the present invention may comprise fucoxanthin or a marine plant extract comprising the same alone or may further comprise one or more pharmaceutically acceptable carrier, excipient or diluent.

The pharmaceutically acceptable carrier further includes an oral administration carrier or parenteral administration carrier. The oral administration carrier includes lactose, starch, cellulose derivative, magnesium, stearate, stearic acid, and the like. Also, the parenteral administration carrier includes water, appropriate oil, saline solution, aqueous glucose, glycol, etc. Additionally, the parenteral administration carrier includes a stabilizer and a preserver. The stabilizer preferably includes antioxidant, such as sodium bisulfate, sodium sulfite and ascorbic acid. The reserver preferably includes benzalkonium chloride, methyl- or propyl-paraben and chloro butanol. Other pharmaceutically acceptable carriers are disclosed in the following reference (Remington's Pharmaceutical Science, 19^{th} Edition, Mack Publishing Company, Easton, Pa., 1995).

The inventive pharmaceutical composition may be administered to any mamalian comprising human being by various routes. For example, it may be administered by oral route or by parenteral route. As for parenteral administration, it may be administered by, but not limited thereto, intravenous, intramuscular, intraarterial, intramarrow, subdural, intracardiac, intracutaneous, subcutaneous, intraperitoneal, intranasal, gastrointestinal tracts, parenteral, sublingual or intrarectal route.

The inventive pharmaceutical composition may be formulated into an oral formulation or a parenteral formulation depending on a selected administration route. In the case of the oral formulation, the inventive pharmaceutical composition may be formulated into powders, granules, tablets, pills, sugar-coated tablets, capsules, liquids, gels, syrups, slurry, suspensions and the like, by a method known in the art. For example, the oral formulation may be obtained as tablets or sugar-coated tablets by blending the active components with a solid excipient, crushing the blend, adding suitable adjuvants, and then processing the mixture into a granular mixture. Examples of suitable excipients may include sugars, including lactose, dextrose, sucrose, sorbitol, mannitol, xylitol, erythritol and maltitol; starches, such as corn starch, wheat starch, rice starch and potato starches; celluloses, such as cellulose, methyl cellulose, sodium carboxymethylcellulose and hydroxypropylmethyl cellulose; and fillers, such as gelatin and polyvinylpyrrolidone. If necessary, a disintegrant, such as crosslinked polyvinylpyrrolidone, agar, alginic acid or sodium alginate, may be used. Furthermore, the inventive pharmaceutical composition may additionally comprise anticoagulants, lubricants, wetting agents, perfume, emulsifiers and/or preservatives. In the case of the parenteral formulation, the inventive pharmaceutical composition may be formulated in the form of injections, cream, lotion, external ointment, oil, moisturizers, gels, aerosols and nasal inhalers, by any method known in the art. The formulation of the above-mentioned is well described in Remington's Pharmaceutical Science, 15th Edition, 1975. Mack Publishing Company, Easton, Pa. 18042, Chapter 87: Blaug, Seymour which is well known prescription book.

The total effective amount of the polypeptide in the inventive composition can be administered to a subject as a single dose, or can be administered using a fractionated treatment protocol, in which the multiple doses are administered over a more prolonged period of time. The amount of the active ingredient in the inventive composition may vary depending on disease severity. In case of parenteral administration, the effective amount of the inventive composition is preferably about 0.01 to 50 mg/kg body weight/day, more preferably 0.1 to 30 mg/kg body weight/day, and, in case of oral administration, the effective amount of the inventive composition is preferably about 0.001 to 100 mg/kg body weight/day, more preferably 0.1 to 50 mg/kg body weight/day with a single dose or multiple doses. However, the effective dose of fucoxanthin or a marine plant extract comprising the same may vary depending on many factors, such as the age, body weight, health condition, sex, disease severity, diet and excretion of a subject in need of treatment, as well as administration time and administration route. In view of these factors, any person skilled in the art may determine an effective dose suitable for the above-described specific use as a treating or a preventing agent for the lipid metabolic disorders of the fucoxanthin or the marine plant extract comprising the same. The inventive composition has no special limitations on its formulation, administration route and administration mode as long as it shows the effects of the present invention.

Meanwhile, the inventive food composition for preventing or improving lipid metabolic disorders is characterized by comprising fucoxanthin or marine plant extract containing the same as an effective ingredient.

The fucoxanthin or marine plant extract containing the same and their activity was described in the above.

The food composition of the present invention comprises all types of food compositions including functional food, nutritional supplement, health food and food additives.

The said food compositions are prepared into various forms by using conventional techniques which are well known in the art. As for health food, for example, but not limited thereto, the fucoxanthin or marine plant extract containing the same may be prepared into tea, juice, and drink for drinking or may be prepared into liquids, granules, capsules, or powder for uptake. Also, conventional active ingredient which is well known as having activity in preventing and treating lipid metabolic disorders may be mixed with fucoxanthin or marine plant extract containing the same of the present invention so as to prepare a composition. Also, for preparing functional foods, but not limited thereto, beverages(including alcoholic beverages), fruits, and their processed foods(e.g. canned fruit, bottled fruit, jam, marmalade etc.), fishes, meats, and their processed foods (e.g. ham, sausage, corn beef etc.), breads and noodles(e.g. Japanese noodle, buckwheat noodle, Ramyen, spaghetti, macaroni etc.), fruit juice, drinks, cookies, toffee, dairy products(e.g. butter, cheese etc.), vegetable oil, margarine, vegetable protein, retort food, frozen food, various seasonings (e.g. soybean paste, soybean sauce, sauce etc.) may be prepared by adding fucoxanthin or marine plant extract containing the same.

In addition, the inventive fucoxanthin or marine plant extract containing the same may be prepared in a form of powder or extract for food additives.

The inventive fucoxanthin or marine plant extract containing the same may be properly combined by the form of composition for food preferably in the range of 0.001 to 50 weight % based on the total weight of a food. More prefeably, the food composition comprising the inventive fucoxanthin or marine plant extract containing the same as an effective ingredient, particularly may be prepared into forms of healthy food by mixing conventional active ingredient which is well known as having activity in preventing and treating lipid metabolic disorders.

A feed composition according to the present invention comprises fucoxanthin or a marine plant extract comprising the same.

The feed composition of the present invention may be prepared into various forms including fermented feedstuff, assorted feed, pellet, silage, etc. The fermented feedstuff may be prepared by adding fucoxanthin or a marine plant extract comprising the same along with various bacteria or enzymes to an organic matter. The assorted feed may be prepared by mixing various common feedstuffs with fucoxanthin or a marine plant extract comprising the same. The pellet type feedstuff may be prepared by pelletizing the fermented feedstuff or the assorted feed using a pelletizer. The silage may be prepared by mixing forage with fucoxanthin or a marine plant extract comprising the same, and fermenting the same through a common method.

The present invention further provides an use of fucoxanthin or a marine plant extract comprising the same for the preparation of an agent for the treatment of lipid metabolic disorders.

Because fucoxanthin or a marine plant extract comprising the same is effective in reducing weight increase and reducing triglyceride and cholesterol level in liver tissue or plasma through inhibiting the synthesis of fatty acid and promoting the oxidation of fatty acid, as described above, it can be effectively used for the preparation of an agent for the treatment of lipid metabolic disorders.

The present invention further provides an use of fucoxanthin or a marine plant extract comprising the same for the preparation of a food composition.

Because fucoxanthin or a marine plant extract comprising the same provides the aforesaid effects, it can be effectively used for the preparation of a food composition for the preventing or improving lipid metabolic disorders.

The present invention further provides an use of fucoxanthin or a marine plant extract comprising the same for the preparation of a feed composition.

The fucoxanthin or a marine plant extract comprising the same may be prepared into various forms including fermented feedstuff, assorted feed, pellet, silage, etc.

In another aspect, the present invention provides a method for preventing or treating lipid metabolic disorders comprising administering fucoxanthin or a marine plant extract comprising the same to a subject in need thereof an effective amount.

As used herein, the term “subject in need thereof” means mammals which need treatment or prevention of lipid metabolic disorders, preferbly human beings.

As used herein, the “effective amount” refers to the amount effective in treating or preventing lipid metabolic disorders.

Administration method and administration doses for administering to a subject in need thereof an effective amount of fucoxanthin or a marine plant extract comprising the same are described well in the above.

In addition, the fucoxanthin or a marine plant extract comprising the same, as described well in the above, is effective in reducing weight increase and reducing triglyceride and cholesterol level in liver tissue or plasma through inhibiting the synthesis of fatty acid and promoting the oxidation of fatty acid.

Therefore, it may be effectively used for the prevention and treatment of lipid metabolic disorders.

### Advantageous Effects

Fucoxanthin or a marine plant extract comprising the same is effective in reducing weight increase and reducing triglyceride and cholesterol level in liver tissue or plasma through inhibiting the synthesis of fatty acid and promoting the oxidation of fatty acid. Therefore, a composition comprising fucoxanthin or a marine plant extract comprising the same of the present invention as an effective ingredient may be effectively used for the prevention and treatment of lipid metabolic disorders.

### MODE FOR INVENTION

Hereinafter, the present invention will be described in detail through examples and test examples. However, the following examples and test examples are for the purpose of illustration only, and they do not limit the scope of the present invention.

### Example 1

### Preparation of Fucoxanthin Extract

240 L of spirit and 40 L of water were added to 45 kg of dry wakame powder. After extracting in a 1 t concentration tank (Jeil Machine, Model No. J003) at 25° C. for 6 hours, the obtained extract was pressed with a filter press using 12 sheets of 300 mm×300 mm pads having a pore size of 0.4 μm so as to remove the remnants of the dry wakame. The extract collected from the filter press was concentrated in a concentration tank of 25° C. and 740 mmHg for 3 hours to a volume of 20 L. Then, it was concentrated again in a vacuum concentrator of 60° C. and 50 mmHg for 8 hours. The final concentrate was lyophilized in a lyophilizer at −40° C. for 48 hours to obtain the fucoxanthin extract sample. Calorie and general composition of the prepared fucoxanthin extract are given in the following Table 1.

TABLE 1
Calorie and general composition of fucoxanthin
extract

| | Calorie and |
|---|---|
| | general |
| | composition |
| Calorie (kcal/100 g) | 670.2 kcal |
| Fat (g/100 g) | 70.6 wt % |
| Protein (g/100 g) | 4.4 wt % |
| Carbohydrate (g/100 g) | 4.3 wt % |
| Sodium (g/100 g) | 6 wt % |

Fucoxanthin concentration of the fucoxanthin extract was determined using HPLC. Symmetry C18 (4.6×250 mm, Waters, Ireland) column was used, and detection of fucoxanthin was made at a wavelength of 450 nm in the ultraviolet (UV) region. Mobile phase was a 1:9 (v/v) mixture of hexane and acetone. Elution was carried out for about 15 minutes at a rate of 0.5 mL/min. The fucoxanthin content was calculated with reference to 94% fucoxanthin (CaroteNature, Switzerland) as standard substance. The fucoxanthin concentration of the fucoxanthin extract was 3.5 wt %.

### Example 2

### Preparation of Highly Pure Fucoxanthin

In order to prepare highly pure fucoxanthin, the fucoxanthin extract obtained in Example 1 was placed on Whatman filter paper No. 2, and vibration was applied while pouring hexane with about 2 times the volume of the extract. This filtration process was repeated 2 times in order to remove highly fat-soluble substances. As a result, 7.5 g of a sample containing about 50% of fucoxanthin was obtained. The fucoxanthin-containing sample was dissolved in about 50 mL of acetone. The solution was flown in a 2,000 mL silica gel column (resin: Merck Kieselgel 66; 70-230 mesh, internal diameter 7.5 cm×length 60 cm) at a rate of about 2,000 mL/hour so that fucoxanthin was adsorbed to silica gel. After the sample adsorption was completed, about 2,000 mL of an 8:2 (v/v) mixture solvent of hexane and acetone was flown in the column so as to remove unadsorbed impurities. 6:4 (v/v) eluent of hexane and acetone was flown in the column so as to elute the components adsorbed to silica gel. Subsequently, about 2,000 mL of the eluent was vacuum concentrated to a volume of about 10 mL using a vacuum concentrator, with the temperature inside the evaporation tube not higher than 40° C. After adding about 10 mL of triply distilled water to the resultant concentrate, the concentrate was allowed to stand at −20° C. for about 4 hours to obtain red precipitate. The precipitate was recovered by filtering through Whatman filter paper No. 2, and dried in a vacuum dryer of 40° C. for about 12 hours. 3.6 g of dry substance was obtained. Purity of fucoxanthin in the obtained highly pure fucoxanthin sample was 97.5%.

### Reference Example 1

Management of Test Animals and Composition of Test Diets

Seventy (70) 4-week-old male C57BL/6N/CriBgi mice weighing 14 g were purchased from Orient, and reared in separate cages maintained at 24° C. and relative humidity of 55%, providing light from 08:00 until 20:00. The mice were accustomed while providing a pellet type diet for a week. The mice, which weighed 18.5-18.7 g, were grouped into seven groups by the randomized block design. Each group was given a different diet, as follows.

Test diets were as follows: “normal diet group”=AIN-76 semisynthetic diet of Teklad (Madison, Wl, USA); “high fat diet group”=normal diet+10% of corn oil and 10% of lard; “test group I”=high fat diet+fucoxanthin extract of Example 1 (fucoxanthin content=0.05%); “test group II”=high fat diet+fucoxanthin extract of Example 1 (fucoxanthin content=0.2%); “test group III”=high fat diet+fucoxanthin extract of Example 2 (fucoxanthin content=0.05%); “test group IV”=high fat diet+fucoxanthin extract of Example 2 (fucoxanthin content=0.2%). The 6 diets (normal diet, high fat diet, and test I, II, III and IV) were given to the animals for 6 weeks. Diet intake was recorded every day, and body weight was measured once a week. Composition of the test diets is given in the following Table 2.

TABLE 2
Composition of test diets (%)

| | Normal | High fat | Test | Test | Test | Test |
|---|---|---|---|---|---|---|
| | diet | diet | I | II | III | IV |
| Casein | 20 | 20 | 20 | 20 | 20 | 20 |
| D-, L- | 0.3 | 0.3 | 0.3 | 0.3 | 0.3 | 0.3 |
| Methionine | | | | | | |
| Corn starch | 15 | — | — | — | — | — |
| Sucrose | 50 | 50 | 48.57 | 44.28 | 49.95 | 49.79 |
| Cellulose | 5 | 5 | 5 | 5 | 5 | 5 |
| Mineral | 3.5 | 3.5 | 3.5 | 3.5 | 3.5 | 3.5 |
| mixture | | | | | | |
| Vitamin | 1 | 1 | 1 | 1 | 1 | 1 |
| mixture | | | | | | |
| Choline | 0.2 | 0.2 | 0.2 | 0.2 | 0.2 | 0.2 |
| bitartrate | | | | | | |
| Corn oil | 5 | 10 | 10 | 10 | 10 | 10 |
| Lard | — | 10 | 10 | 10 | 10 | 10 |
| Fucoxanthin or | — | — | 1.43 | 5.72 | 0.05 | 0.21 |
| wakame extract | | | | | | |
| comprising | | | | | | |
| fucoxanthin | | | | | | |
| Total (%) | 100 | 100 | 100 | 100 | 100 | 100 |

### Test Example 1

### Effect of Reducing Body Weight Increase of Highly Pure Fucoxanthin and Fucoxanthin Extract

It was observed whether the test groups fed with the fucoxanthin extract or the highly pure fucoxanthin of Example 1 or Example 2 exhibited less weight increase as compared to the control group fed only with the high fat diet. During the 6-week test period, body weight was measured every week. Significance of differences of means of the groups was evaluated by one-way analysis of variance (ANOVA) technique. Duncan's multiple range test was employed for post-evaluation of the groups, with P<0.05. The result (mean standard±deviation) is given in the following Table 3.

TABLE 3
Effect of reducing body weight increase of highly pure fucoxanthin and fucoxanthin extract

| Diet groups | Week 0 | Week 1 | Week 2 | Week 3 | Week 4 | Week 5 | Week 6 |
|---|---|---|---|---|---|---|---|
| Normal diet | 18.58 ± 0.4 | 20.50 ± 0.4 | 21.87 ± 0.2 | 23.08 ± 0.4^{ab} | 24.91 ± 0.6^{ab} | 26.67 ± 0.5^{b} | 27.82 ± 0.6^{b} |
| High fat diet | 18.65 ± 0.3 | 20.50 ± 0.3 | 22.20 ± 0.3 | 23.79 ± 0.3^{a} | 25.97 ± 0.4^{a} | 28.18 ± 0.4^{a} | 30.46 ± 0.3^{a} |
| Test I | 18.61 ± 0.3 | 20.39 ± 0.3 | 22.20 ± 0.4 | 22.60 ± 0.4^{b} | 24.43 ± 0.5^{b} | 26.16 ± 0.6^{b} | 27.68 ± 0.6^{b} |
| Test II | 18.61 ± 0.3 | 20.13 ± 0.2 | 21.79 ± 0.2 | 22.83 ± 0.2^{ab} | 24.46 ± 0.4^{b} | 25.78 ± 0.3^{b} | 26.07 ± 0.4^{c} |
| Test III | 18.56 ± 0.3 | 20.19 ± 0.2 | 21.79 ± 0.3 | 22.70 ± 0.2^{b} | 24.39 ± 0.3^{b} | 26.46 ± 0.3^{b} | 27.80 ± 0.3^{b} |
| Test IV | 18.58 ± 0.3 | 20.19 ± 0.3 | 22.30 ± 0.3 | 22.96 ± 0.4^{ab} | 25.17 ± 0.5^{ab} | 26.39 ± 0.6^{b} | 27.37 ± 0.7^{bc} |

^{a,b,c}Groups denoted with the same characters are statistically insignificant from each other (P < 0.05).

As shown in Table 3, all the test groups fed with the highly pure fucoxanthin or the fucoxanthin extract exhibited lower weight increase as compared to the control group fed only with the high fat diet. Accordingly, the fucoxanthin extract can effectively inhibit the increase of body weight, and may be used effectively for a composition for the prevention and treatment of metabolic disorders, in particular, obesity.

### Test Example 2

### Effect of Reducing Production of Triglyceride and Cholesterol of Highly Pure Fucoxanthin and Fucoxanthin Extract

<2-1> Change of Triglyceride and Cholesterol Level in Liver Tissue

Livers were taken from the mice which were fed with the test diets of Reference Example 1 for 6 weeks, rinsed several times in phosphate buffered saline (PBS) solution, and then dried and weighed. 0.2 g of liver tissue was homogenized in 3 mL of chloroform:methanol (2:1) solution to extract lipid, and extraction was carried out 3 more times using an equal amount of extraction solvent. The extract was filtered through Whatman filter paper No. 2, dried with nitrogen gas, and dissolved again in 1 mL of the same extraction solvent. 100 μL was completely dried using nitrogen gas. Then, 5 mL of ethanol was added and the level of cholesterol and triglyceride was quantitated.

Specifically, cholesterol level was measured using an enzymatic kit (Asan kit, Korea). Because cholesterol exists in two forms—cholesteryl ester (CE) and free cholesterol—CE was converted to fatty acid and free cholesterol using cholesterol esterase in order to quantitate both. The converted free cholesterol was converted to Δ4-cholestenone by treating with cholesterol oxidase. Hydrogen peroxide produced in the process was turned to red by mixing with peroxidase, phenol and 4-amino-antiptrine. Absorbance was measured at 500 nm and the result was compared with that of cholesterol standard solution (300 mg/dL). The result is given in the following Table 4.

Triglyceride level was assayed measured using an enzymatic kit (Asan kit, Korea). Triglyceride was hydrolyzed by lipase into glycerin and fatty acid, and converted to L-α-phosphoglycerol by adding ATP and glycerol kinase (GK). The converted L-α-phosphoglycerol was reacted to produce hydrogen peroxide by adding oxygen (O₂) and glycerophospho oxidase. The produced hydrogen peroxide was turned to red by mixing with peroxidase and 4-amino-antiptrine. Absorbance was measured at 550 nm and the result was compared with that of cholesterol standard solution (300 mg/dL). The result is given in Table 4.

TABLE 4
Change of triglyceride and cholesterol level
in liver tissue

| | Triglyceride level | Cholesterol level |
|---|---|---|
| | in liver tissue | in liver tissue |
| | (mmol/g liver) | (mmol/g liver) |
| Normal diet | 0.19 ± 0.03^{b} | 2.19 ± 0.38^{b} |
| High fat diet | 0.22 ± 0.04^{a} | 2.38 ± 0.69^{a} |
| Test I | 0.12 ± 0.01^{c} | 1.88 ± 0.33^{b} |
| Test II | 0.11 ± 0.02^{c} | 1.88 ± 0.17^{b} |
| Test III | 0.14 ± 0.01^{c} | 1.77 ± 0.27^{b} |
| Test IV | 0.17 ± 0.02^{b} | 1.96 ± 0.23^{b} |

^{a, b, c}Groups denoted with the same characters are statistically insignificant from each other (P < 0.05)

As shown in Table 4, the control group fed with the high fat diet exhibited significantly higher triglyceride and cholesterol level in liver tissue as compared to the normal diet group. In contrast, the test group fed with highly pure fucoxanthin or fucoxanthin extract exhibited significantly lower triglyceride and cholesterol level in liver tissue, comparable to the normal diet group. Accordingly, the fucoxanthin extract or the highly pure fucoxanthin reduces triglyceride and cholesterol level in liver tissue, and may be used effectively for a composition for the prevention and treatment of lipid metabolic disorders, in particular, fatty liver.

<2-2> Change of Triglyceride and Cholesterol Level in Plasma

The mice fed with the test diets for 6 weeks of Reference Example 1 were fasted for 12 hours. The mice were anesthetized first by ether inhalation, and then by intramuscular injection of ketamine-HCl (Yuhan). Then, blood was taken from the abdominal inferior vena cava and collected in a heparin-treated test tube. Plasma was separated by carrying out centrifuge at 3,000 rpm for 15 minutes. The separated plasma was stored at −70° C. Total cholesterol and triglyceride level in the plasma was quantitated in the same manner as Test Example 2-1. The result is given in the following Table 5.

TABLE 5
Change of triglyceride and cholesterol level
in plasma

| | Triglyceride level | Cholesterol level |
|---|---|---|
| | in plasma (mmol/L) | in plasma (mmol/L) |
| Normal diet | 1.26 ± 0.11^{b} | 2.30 ± 0.00^{b} |
| High fat diet | 1.56 ± 0.08^{a} | 2.71 ± 0.05^{a} |
| Test I | 1.16 ± 0.08^{b} | 2.12 ± 0.14^{b} |
| Test II | 1.02 ± 0.08^{b} | 2.17 ± 0.12^{a} |
| Test III | 1.15 ± 0.07^{b} | 2.35 ± 0.22^{a} |
| Test IV | 1.24 ± 0.07^{b} | 2.35 ± 0.11^{b} |

^{a, b, c}Groups denoted with the same characters are statistically insignificant from each other (P < 0.05).

As shown in Table 5, the control group fed only with the high fat diet exhibited significantly higher triglyceride and cholesterol level in plasma as compared to the normal diet group. In contrast, the test group fed with highly pure fucoxanthin or fucoxanthin extract exhibited significantly lower triglyceride and cholesterol level in plasma, comparable to the normal diet group. Accordingly, the highly pure fucoxanthin or the fucoxanthin extract reduces triglyceride and cholesterol level in plasma, and may be used effectively for a composition for the prevention and treatment of lipid metabolic disorders, in particular, hyperlipidemia.

<2-3> Change of Cholesterol in Feces

Feces were taken from the mice fed with the test diets for 6 weeks of Reference Example 1. The amount of feces and cholesterol content in the feces were measured. The result is given in the following Table 6.

TABLE 6
Change of amount of feces and cholesterol
content in feces

| | | Cholesterol |
|---|---|---|
| | Amount of feces | content in feces |
| | and (g/day) | (umol/g) |
| Normal diet | 0.134 ± 0.00^{a} | 97.77 ± 2.03^{a} |
| High fat diet | 0.140 ± 0.00^{a} | 111.08 ± 2.02^{a} |
| Test I | 0.191 ± 0.00^{b} | 307.64 ± 25.86^{c} |
| Test II | 0.231 ± 0.01^{c} | 766.75 ± 30.03^{b} |
| Test III | 0.226 ± 0.01^{c} | 237.88 ± 10.39^{c} |
| Test IV | 0.209 ± 0.01^{bc} | 508.79 ± 13.25^{c} |

^{a, b, c}Groups denoted with the same characters are statistically insignificant from each other (P < 0.05).

As shown in Table 6, the test group fed with the highly pure fucoxanthin or the fucoxanthin extract exhibited significantly higher cholesterol content in feces as compared to the control group fed with the high fat diet group. Accordingly, the highly pure fucoxanthin or the fucoxanthin extract inhibits the intake of cholesterol, and may be used effectively for a composition for the prevention and treatment of lipid metabolic disorders.

### Test Example 3

### Effect of Inhibiting Synthesis of Fatty Acid of Fucoxanthin Extract

<3-1> Effect of Inhibiting Synthesis of Fatty Acid in Adipose Tissue

0.5 g of adipose tissue was taken from the mice anesthetized in Test Example 2-2, lysed using a buffer solution (Glascol, 099CK44, USA) containing 0.1 M triethanolamine, 0.02 M ethylenediaminetetracetate (EDTA, pH 7.4) and 0.002 M dithiothreitol (DTT), and centrifuged at 10,000×g for 15 minutes. The supernatant was centrifuged again at 12,000×g for 15 minutes. Then, the supernatant was subjected to high-speed centrifuging (Beckman, Optima TLX-120, USA) at 100,000×g for 1 hour. Then, change of the activity of enzymes involved in the synthesis of fatty acid—fatty acid synthase (FAS), glucose-6-phosphate dehydrogenase (G6PD) and malic enzyme (ME)—was measured.

Specifically, FAS activity was determined as follows. 500 μM buffer solution (potassium phosphate buffer, pH 7.0), 33 nM acetyl-CoA, 100 nM NADPH, 1 μM β-mercaptoethanol and cytosol fraction were mixed. After 10 minutes of reaction at 30° C., decrease of absorbance was measured. FAS activity was calculated as nmol of NADPH oxidized per 1 mg of protein in cell per minute.

The G6PD enzyme is an enzyme that supplied reducing energy required for the synthesis of fatty acid. That is, it converts NADH to NADPH, and is one of the enzymes involved in the synthesis of fatty acid. G6PD activity was measured at 340 nm by the degree of reduction of NADP⁺ to NADPH by G6PD. Specifically, 40 μL of 6 mM NADP⁺, 40 μL of 0.1 M glucose-6-phosphate and 20 μL of G6PD were sequentially added to 900 μL of 55 mM Tris-HCl (pH 7.8) containing 3.3 mM magnesium chloride (MgCl₂), and change of absorbance was measured at 340 nm (25° C.) for 90 seconds. G6PD activity was calculated as nmol of NADPH produced per 1 mg of protein in cell per minute.

The ME enzyme is also an enzyme that supplied reducing energy required for the synthesis of fatty acid. It converts NADH to NADPH, and is one of the enzymes involved in the synthesis of fatty acid. ME activity was determined as follows. An enzyme solution was added to 1 mL of a reaction solution containing 0.4 M triethanolamine (pH 7.4), 30 mM malic acid, 0.12 M magnesium chloride and 3.4 mM NADP. After 2 minutes of reaction at 27° C., absorbance was measured at 340 nm. ME activity was calculated as nmol of NADPH produced per 1 mg of protein in cell per minute.

Activity of the aforesaid enzymes involved in the synthesis of fatty acid in adipose tissue is given in the following Table 7.

TABLE 7
Change of activity of enzymes involved in
synthesis of fatty acid in adipose tissue (P < 0.05)

| | ME (mmol/ | FAS | G6PD |
|---|---|---|---|
| | min/mg | (mmol/min/mg | (mmol/min/mg |
| | protein) | protein) | protein) |
| Normal | 2.06 ± 0.32^{b} | 196.52 ± 34.30^{c} | 1.66 ± 0.19^{a} |
| diet | | | |
| High fat | 1.69 ± 0.21^{b} | 145.93 ± 18.21^{b} | 2.81 ± 0.33^{b} |
| diet | | | |
| Test I | 1.04 ± 0.15^{a} | 105.63 ± 20.96^{ab} | 1.15 ± 0.17^{a} |
| Test II | 0.94 ± 0.19^{a} | 106.45 ± 8.14^{ab} | 1.14 ± 0.19^{a} |
| Test III | 0.92 ± 0.14^{a} | 90.55 ± 9.66^{a} | 1.25 ± 0.27^{a} |
| Test IV | 0.94 ± 0.11^{a} | 74.26 ± 6.38^{a} | 1.27 ± 0.15^{a} |

^{a, b, c}Groups denoted with the same characters are statistically insignificant from each other.

As shown in Table 7, the test groups fed with the fucoxanthin extract exhibited significantly lower FAS, ME and G6PD activity in adipose tissue than the control group fed only with the high fat diet. The test groups fed with the highly pure fucoxanthin showed decreased FAS activity and significantly decreased ME and G6PD activity. Accordingly, the highly pure fucoxanthin or the fucoxanthin extract reduces the activity of the enzymes involved in the synthesis of fatty acid in adipose tissue, and may be used effectively for a composition for the prevention and treatment of lipid metabolic disorders.

<3-2> Effect of Inhibiting Synthesis of Fatty Acid in Liver Tissue

0.5 g of liver tissue taken from the mice anesthetized in Test Example 2-2 was treated in the same manner as Example 3-1. Change of activity of FAS, ME and G6PD was measured, and the result is given in the following Table 8.

TABLE 8
Change of activity of enzymes involved in
synthesis of fatty acid in liver tissue (P < 0.05)

| | | FAS | G6PD |
|---|---|---|---|
| | ME (mmol/min/mg | (mmol/min/mg | (mmol/min/mg |
| | protein) | protein) | protein) |
| Normal diet | 145.69 ± 22.23^{b} | 9.20 ± 1.32^{ab} | 131.71 ± 26.24^{c} |
| High fat diet | 153.88 ± 8.72^{b} | 12.61 ± 0.72^{c} | 79.94 ± 5.77^{b} |
| Test I | 74.31 ± 5.85^{a} | 10.63 ± 0.91^{abc} | 47.34 ± 6.51^{a} |
| Test II | 73.09 ± 8.03^{a} | 8.53 ± 1.18^{ab} | 48.48 ± 4.06^{a} |
| Test III | 77.06 ± 7.08^{a} | 7.92 ± 0.59^{a} | 51.53 ± 4.31^{a} |
| Test IV | 73.91 ± 6.37^{a} | 9.49 ± 0.65^{ab} | 50.51 ± 3.74^{a} |

^{a, b, c}Groups denoted with the same characters are statistically insignificant from each other.

As shown in Table 8, the test groups fed with the highly pure fucoxanthin or the fucoxanthin extract exhibited significantly lower FAS activity in liver tissue than the control group fed only with the high fat diet. The test groups fed with the highly pure fucoxanthin or the fucoxanthin extract showed significantly decreased ME and G6PD activity as compared to the control group fed only with the high fat diet. Accordingly, the highly pure fucoxanthin or the fucoxanthin extract reduces the activity of the enzymes involved in the synthesis of fatty acid in liver tissue, and may be used effectively for a composition for the prevention and treatment of lipid metabolic disorders.

### Test Example 4

### Effect of Inhibiting Synthesis of Triglyceride of Highly Pure Fucoxanthin and Fucoxanthin Extract

Activity of the enzyme phosphatidate phosphohydrolase (PAP), which is an enzyme that catalyzes the synthesis of triglyceride in liver tissue by converting fatty acid and 1,3-diglyceride to triglyceride through the phosphatidic acid pathway, was measured as follows.

Specifically, 50 μL of a substrate in which 1 mM phosphatidate and phosphatidylcholine were dissolved in 0.9% NaCl solution was added to 50 μL of a reaction solution containing 0.05 M Tris-HCl (pH 7.0), 1.25 mM EDTA and 1.0 mM magnesium chloride (MgCl₂). Then, 0.1 mL of PAP was added and reaction was carried out at 37° C. 15 minutes later, 0.1 mL of 1.8 M sulfuric acid (H₂SO₄) was added to stop the reaction. Then, 0.25 mL of 1.25% ascorbic acid, 0.25 mL of 0.32% ammonium molybdate and 0.1 mL of 0.13% sodium dodecyl sulfate were added. After heat treating at 45° C. for 20 minutes, absorbance was measured at 820 nm. The result is given in the following Table 9.

TABLE 9
Change of activity of enzyme involved in
synthesis of triglyceride

| | PAP |
|---|---|
| | (mmol/min/mg protein) |
| Normal diet | 28.86 ± 1.83^{ab} |
| High fat diet | 31.46 ± 4.78^{b} |
| Test I | 25.15 ± 1.76^{ab} |
| Test II | 22.89 ± 1.39^{a} |
| Test III | 21.62 ± 1.54^{a} |
| Test IV | 26.14 ± 1.03^{ab} |

^{a, b, c}Groups denoted with the same characters are statistically insignificant from each other.

As shown in Table 9, the control group fed only with the high fat diet exhibited higher PAP activity in liver tissue than the normal diet group. In contrast, the test groups fed with the highly pure fucoxanthin or the fucoxanthin extract exhibited decreased PAP activity in liver tissue. Accordingly, the highly pure fucoxanthin or the fucoxanthin extract reduces the activity of the enzyme involved in the synthesis of triglyceride, and may be used very effectively for the prevention and treatment of lipid metabolic disorders.

### Test Example 5

### Effect of Promoting Oxidation of Fatty Acid of Highly Pure Fucoxanthin and Fucoxanthin Extract

<5-1> Change of Activity of Carnitine Palmitoyltransferase (CPT)

CPT is an enzyme involved in the oxidation of fatty acid and may be used as an index for the degree of oxidation of fatty acid. CPT activity was calculated from the measurement of CoASH produced from palmitoyl-CoA using 5,5-dithiobis-(2-nitrobenzoic acid) (DTNB). Specifically, 50 μL of mitochondrial fraction was added to a reaction solution containing 116 mM Tris-HCl (pH 8.0), 1.1 mM EDTA, 2.50 mM 1-carnitine, 0.5 mM DTNB, 75 mM palmitoyl-CoA and 0.2% Triton X-100 to initiate reaction. Then, change of absorbance was measured at 25° C. and 412 nm for 2 minutes. The result is given in the following Table 10.

TABLE 10
Change of activity of CPT

| | CPT |
|---|---|
| | (mmol/min/mg protein) |
| Normal diet | 13.00 ± 1.91^{ab} |
| High fat diet | 12.92 ± 1.26^{ab} |
| Test I | 29.70 ± 6.94^{bc} |
| Test II | 37.28 ± 12.05^{c} |
| Test III | 13.36 ± 1.66^{ab} |
| Test IV | 40.30 ± 12.10^{c} |

^{a, b, c}Groups denoted with the same characters are statistically insignificant from each other.

As shown in Table 10, the control group fed only with the high fat diet exhibited lower CPT activity than the normal diet group. In contrast, the test groups fed with the highly pure fucoxanthin or the fucoxanthin extract exhibited increased CPT activity. Accordingly, the highly pure fucoxanthin or the fucoxanthin extract reduces the activity of the enzyme involved in the oxidation of fatty acid, and may be used very effectively for the prevention and treatment of lipid metabolic disorders.

<5-2> Change of β-Oxidation Activity

Mitochondrial β-oxidation activity was measured from the degree of reduction of NAD to NADH using palmitoyl-CoA. Specifically, 10 μL of mitochondrial fraction was added to a reaction solution containing 50 mM Tris-HCl (pH 8.0), 20 mM NAD, 0.33 M DTT, 1.5% BSA (1.5 g/100 mL), 2% Triton X-100 (2 g/100 mL), 10 mM CoA, 1 mM FAD, 100 mM KCN and 5 mM palmitoyl-CoA to initiate reaction. Then, change of absorbance was measured at 37° C. and 340 nm for 5 minutes. β-Oxidation activity was calculated as nmol of NADH produced per 1 mg of mitochondrial protein per minute. The result is given in the following Table 11.

TABLE 11
Change of β-oxidation activity (P < 0.05)

| | β-Oxidation activity |
|---|---|
| | (mmol/min/mg protein) |
| Normal diet | 2.32 ± 0.50^{ab} |
| High fat diet | 2.22 ± 0.52^{ab} |
| Test I | 13.17 ± 4.31^{c} |
| Test II | 8.48 ± 2.61^{abc} |
| Test III | 9.46 ± 2.56^{bc} |
| Test IV | 13.28 ± 3.79^{c} |

^{a, b, c}Groups denoted with the same characters are statistically insignificant from each other.

As shown in Table 11, the control group fed only with the high fat diet exhibited lower β-oxidation activity than the normal diet group. In contrast, the test groups fed with the highly pure fucoxanthin or the fucoxanthin extract exhibited increased β-oxidation activity. Accordingly, the highly pure fucoxanthin or the fucoxanthin extract promotes β-oxidation, and may be used very effectively for the prevention and treatment of lipid metabolic disorders.

### Test Example 6

### Effect of Highly Pure Fucoxanthin and Fucoxanthin Extract on Expression of mRNA for Fatty Acid Synthase and Oxidase

<6-1> Change of Expression of mRNA for Fatty Acid Synthase and Oxidase in Adipose Tissue

5 mL of Trizol was added to 0.5 g of white adipose tissue. After pulverizing in liquid nitrogen using a mortar, the tissue was added to 1 mL of chloroform. After mixing for 15-30 seconds and placing in ice for 5 minutes, centrifuge was carried out at 12,000×g and 4° C. for 15 minutes. Then, the aqueous phase was separated. After adding 2.5 mL of isopropanol and leaving alone at room temperature for 15 minutes, centrifuge was carried out again at 12,000×g and 4° C. for 5 minutes. After removal of 75% ethanol and drying, the sample was dissolve in DEPC-H₂O and stored at −70° C. The isolated RNA was diluted and absorbance was measured at 260 nm using a UV spectrometer. The status of RNA was confirmed by electrophoresis in agarose gel.

First stand cDNA was synthesized from the isolated RNA through reverse transcription. Specifically, 1 μL of 500 μg/mL oligo(dT) 15 (Invitrogen) and 1 μL of 10 mM dNTP were added to 5 μg of the isolated RNA, and distilled water was added. The resultant solution was heated at 65° C. for 5 minutes and cooled in ice. Then 4 μL of 5× buffer (250 mM Tris-HCL, pH 8.3, 375 mM KCL, 15 mM MgCl₂) and 2 μl of 0.1 M DTT were added. After heating at 42° C. for 2 minutes, 1 μL (200 units) of reverse transcriptase was added. After performing reaction at 42° C. for 50 minutes followed by heating at 70° C. for 15 minutes, the reaction was stopped by deactivating the reverse transcriptase. After diluting with 3 times the volume of sterilized distilled water, the sample was stored at −70° C.

Thus prepared cDNA was distilled 10-fold, and primers for analyzing the expression of the respective genes (CPT, β-oxidation, FAS, ME and G6PD) were obtained from Genotech (Daejeon, Korea). The reaction solution comprised 10.0 μL of 2×SYBR master mix, 4 μL of template, 400 nM of primer and remainder of distilled water to make 20 μL. The reaction condition was: 2 minutes at 50° C., 10 minutes at 95° C., 15 seconds at 95° C. and 1 minute at 60° C. This cycle was repeated for 40 times. Fluorescence signals were monitored for each cycle and threshold cycle (Cr) was analyzed for quantitative analysis of mRNA for the test groups (Applied Biosystems, SDS7000) (Livak, 2001). GAPDH was used as internal transcription marker. The result is given in the following Table 12.

TABLE 12
Change of expression of mRNA for fatty acid synthase and oxidase in adipose tissue

| | Normal diet | High fat diet | Test I | Test II | Test III | Test IV |
|---|---|---|---|---|---|---|
| CPT | 1.00 ± 0.03^{a} | 1.11 ± 0.17^{ab} | 1.50 ± 0.09^{bc} | 1.69 ± 0.27^{c} | 1.65 ± 0.15^{c} | 1.51 ± 0.06^{bc} |
| β-Oxidation | 1.00 ± 0.13^{ab} | 0.24 ± 0.05^{a} | 2.71 ± 0.16^{c} | 2.33 ± 0.71^{c} | 1.41 ± 0.50^{b} | 1.26 ± 0.24^{b} |
| FAS | 1.00 ± 0.15^{d} | 0.76 ± 0.12^{cd} | 0.71 ± 0.06^{bc} | 0.45 ± 0.08^{ab} | 0.51 ± 0.03^{abc} | 0.28 ± 0.05^{a} |
| ME | 1.00 ± 0.22^{bc} | 1.35 ± 0.20^{c} | 0.84 ± 0.06^{ab} | 0.80 ± 0.05^{ab} | 0.70 ± 0.07^{ab} | 0.52 ± 0.06^{a} |
| G6PD | 1.00 ± 0.11^{abc} | 1.35 ± 0.22^{c} | 0.94 ± 0.04^{abc} | 0.85 ± 0.08^{ab} | 1.10 ± 0.15^{abc} | 0.67 ± 0.04^{a} |

(P < 0.05)
^{a,b,c}Groups denoted with the same characters are statistically insignificant from each other.

As shown in Table 12, the test groups fed with the highly pure fucoxanthin or the fucoxanthin extract exhibited increased expression of mRNA for CPT, which is an oxidase involved in the oxidation of fatty acid, as compared to the control group fed only with the high fat diet. Further, the test groups fed with the highly pure fucoxanthin or the fucoxanthin extract exhibited significantly increased expression of mRNA for β-oxidase in adipose tissue, as compared to the control group fed only with the high fat diet.

And, the test groups fed with the highly pure fucoxanthin or the fucoxanthin extract exhibited decreased expression of mRNA for FAS, a fatty acid synthase, as compared to the control group fed only with the high fat diet. Further, the test groups fed with the highly pure fucoxanthin or the fucoxanthin extract exhibited significantly decreased expression of mRNA for ME, as compared to the control group fed only with the high fat diet. And, the test groups fed with the highly pure fucoxanthin or the fucoxanthin extract exhibited significantly decreased expression of mRNA for G6PD, as compared to the control group fed only with the high fat diet.

Accordingly, the highly pure fucoxanthin or the fucoxanthin extract promotes the oxidation of fatty acid and inhibits the synthesis of fatty acid, and may be used very effectively for the prevention and treatment of lipid metabolic disorders.

<6-2> Change of Expression of mRNA for Fatty Acid Synthase and Oxidase in Liver Tissue

RNA was isolated from the liver tissue and cDNA was synthesized therefrom in the same manner as Test Example 6-1. Through real-time PCR, expression of mRNA for peroxisome proliferator-activated receptor α (PPARα), lipoprotein lipase (LPL) and ME was analyzed. The result is given in the following Table 13.

TABLE 13
Change of expression of mRNA for fatty acid synthase and oxidase in liver tissue

| | Normal diet | High fat diet | Test I | Test II | Test III | Test IV |
|---|---|---|---|---|---|---|
| PPARα | 1.00 ± 0.29^{a} | 0.65 ± 0.11^{a} | 0.85 ± 0.06^{a} | 1.57 ± 0.08^{b} | 1.22 ± 0.08^{ab} | 1.57 ± 0.36^{b} |
| LPL | 1.00 ± 0.06^{a} | 1.17 ± 0.08^{a} | 1.22 ± 0.05^{a} | 1.84 ± 0.14^{b} | 2.11 ± 0.09^{b} | 1.20 ± 0.14^{a} |
| ME | 1.00 ± 0.06^{c} | 0.89 ± 0.08^{c} | 0.64 ± 0.04^{ab} | 0.88 ± 0.10^{c} | 0.84 ± 0.08^{bc} | 0.53 ± 0.03^{a} |

(P < 0.05)
^{a,b,c}Groups denoted with the same characters are statistically insignificant from each other.

As shown in Table 13, the test groups fed with the highly pure fucoxanthin or the fucoxanthin extract exhibited increased expression of mRNA for PPARα, which is involved in the oxidation of fatty acid, as compared to the control group fed only with the high fat diet. Further, the test groups fed with the highly pure fucoxanthin or the fucoxanthin extract exhibited increased expression of mRNA for LPL, which hydrolyzes triglyceride, as compared to the control group fed only with the high fat diet. And, the test groups fed with the highly pure fucoxanthin or the fucoxanthin extract exhibited decreased expression of mRNA for ME, which is involved in the synthesis of fatty acid, as compared to the control group fed only with the high fat diet.

Accordingly, the highly pure fucoxanthin or the fucoxanthin extract promotes the oxidation of fatty acid and inhibits the synthesis of fatty acid, and may be used very effectively for the prevention and treatment of lipid metabolic disorders.

### Preparation Example 1

### Powder

The following ingredients were mixed and filled in an airtight bag according to common method to prepare powder:

| | |
|---|---|
| Fucoxanthin extract of Example 2 | 50 mg |
| Crystalline cellulose | 2 g |

### Preparation Example 2

### Tablet I

The following ingredients were mixed and prepared into tablet according to common method:

| | |
|---|---|
| Fucoxanthin extract of Example 2 | 50 mg |
| Crystalline cellulose | 400 mg |
| Magnesium stearate | 5 mg |

### Preparation Example 3

### Tablet II

The following ingredients were mixed and prepared into tablet according to common method:

| | |
|---|---|
| Fucoxanthin extract of Example 1 | 400 mg |
| Crystalline cellulose | 100 mg |
| Magnesium stearate | 5 mg |

### Preparation Example 4

### Tablet III

55 wt % of Spirulina, 10 wt % of guar gum enzyme hydrolysate, 0.01 wt % of vitamin B₁ hydrochloride, 0.01 wt % of vitamin B₆ hydrochloride, 0.23 wt % of D-, L-methionine, 0.7 wt % of magnesium stearate, 22.2 wt % of lactose and 1.85 wt % of cornstarch were mixed with 10 wt % of fucoxanthin extract of Example 1, prepared into tablet according to common method.

### Preparation Example 5

### Capsule I

The following ingredients were mixed and filled into gelatin capsule according to common method to prepare capsule:

| | |
|---|---|
| Fucoxanthin extract of Example 2 | 30 mg |
| Whey protein | 100 mg |
| Crystalline cellulose | 400 mg |
| Magnesium stearate | 6 mg |

### Preparation Example 6

### Capsule II

The following ingredients were mixed and filled into gelatin capsule according to common method to prepare capsule:

| | |
|---|---|
| Fucoxanthin extract of Example 1 | 300 mg |
| Cornstarch | 100 mg |
| Crystalline cellulose | 100 mg |
| Magnesium stearate | 5 mg |

### Preparation Example 7

### Injection

The active ingredient was dissolved in distilled water for injection according to common method. After adjusting pH to about 7.5, the remaining ingredients were dissolved in distilled water for injection and filled in a 2 mL ampule followed by sterilization:

| | |
|---|---|
| Fucoxanthin extract of Example 2 | 100 mg |
| Water for injection | adequate |
| pH adjuster | adequate |

### Preparation Example 8

### Sunsik

Brown rice, barley, glutinous rice and adlay were converted to alpha-starch, dried, and ground into 60 mesh powder according to common method. Black bean, black sesame and wild sesame were dried and ground into 60 mesh powder according to common method. Thus prepared powder of grains and seeds was mixed with the fucoxanthin extract of Example 1 as follows.

Grains: black rice 30 wt %, adlay 15 wt %, barley 20 wt %, glutinous rice 9 wt %

Seeds: wild sesame 7 wt %, black bean 8 wt %, black sesame 7 wt %

Fucoxanthin extract of Example 13 wt %, yeongji 0.5 wt %, foxglove 0.5 wt %<

### Preparation Example 9

### Chewing Gum

20 wt % of gum base, 76.9 wt % of sugar, 1 wt % of fragrance and 2 wt % of water were mixed with 0.1 wt % of the fucoxanthin extract of Example 1 and prepared into chewing gum according to common method.

### Preparation Example 10

### Candy

60 wt % of sugar, 39.8 wt % of starch syrup and 0.1 wt % of fragrance were mixed with 0.1 wt % of the fucoxanthin extract of Example 1 and prepared into candy according to common method.

### Preparation Example 11

### Biscuit

25.59 wt % of weak flour, 22.22 wt % of medium flour, 4.80 wt % of refined sugar, 0.73 wt % of table salt, 0.78 wt % of glucose, 11.78 wt % of palm shortening oil, 1.54 wt % of ammonium, 0.17 wt % of baking soda, 0.16 wt % of sodium bisulfite, 1.45 wt % of rice flour, 0.0001 wt % of vitamin B₁, 0.0001 wt % of vitamin B₂, 0.04 wt % of milk flavor, 20.6998 wt % of water, 1.16 wt % of whole milk powder, 0.29 wt % of milk replacer, 0.03 wt % of monobasic calcium phosphate, 0.29 wt % of sulfonate and 7.27 wt % of spray milk were mixed with 1 wt % of the fucoxanthin extract of Example 1 and prepared into biscuit according to common method.

### Preparation Example 12

### Drink

0.26 wt % of honey, 0.0002 wt % of thioctic amide, 0.0004 wt % of nicotinamide, 0.0001 wt % of sodium riboflavin hydrochloride, 0.0001 wt % of pyridoxine hydrochloride, 0.001 wt % of inositol, 0.002 wt % of orotic acid and 98.7362 wt % of water were mixed with 1 wt % of the fucoxanthin extract of Example 1 and prepared into health drink according to common method.

### INDUSTRIAL APPLICABILITY

Fucoxanthin or a marine plant extract comprising the same is effective in reducing weight increase and reducing triglyceride and cholesterol level in liver tissue, or plasma through inhibiting the synthesis of fatty acid and promoting the oxidation of fatty acid. Therefore, the composition comprising fucoxanthin or a marine plant extract comprising the same as an effective ingredient may be effectively used for the prevention and treatment of lipid metabolic disorders.

1. A pharmaceutical composition for preventing or treating lipid metabolic disorders comprising fucoxanthin of Chemical Formula 1 or a marine plant extract comprising the same as an effective ingredient, wherein the lipid metabolic disorders are selected from the group consisting of diabetes, fatty liver, hyperlipidemia, arteriosclerosis, atherosclerosis, hypertension, cerebral apoplexy and myocardial infarction.

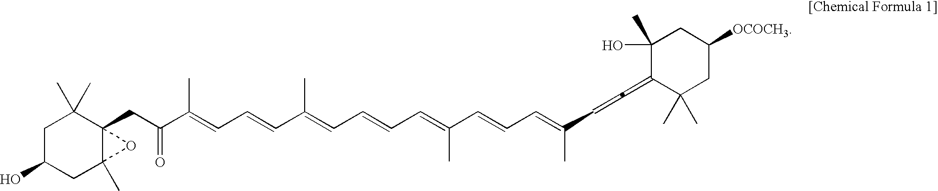

2. A food composition for preventing or improving lipid metabolic disorders comprising fucoxanthin of Chemical Formula 1 or a marine plant extract comprising the same as an effective ingredient, wherein the lipid metabolic disorders are selected from the group consisting of diabetes, fatty liver, hyperlipidemia, arteriosclerosis, atherosclerosis, hypertension, cerebral apoplexy and myocardial infarction.

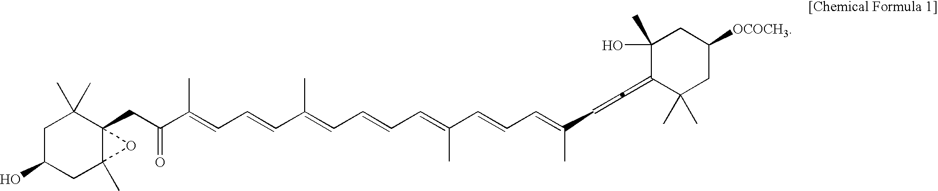

3. A feed composition preventing or improving lipid metabolic disorders comprising fucoxanthin of Chemical Formula 1 or a marine plant extract comprising the same as an effective ingredient, wherein the lipid metabolic disorders are selected from the group consisting of diabetes, fatty liver, hyperlipidemia, arteriosclerosis, atherosclerosis, hypertension, cerebral apoplexy and myocardial infarction.

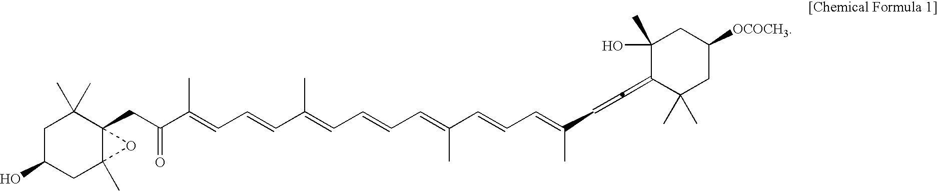

4. The composition of claim 1, wherein the marine plant are one or more plant which are selected from the group consisting of wakame, dashima, gulfweed and hijiki.

5. The composition of claim 1, wherein the marine plant extract is obtained by extracting marine plants with water, spirit, hexane, ethyl acetate, isopropyl alcohol, acetone or a mixture thereof at 10-50° C. for 1-48 hours.

6. (canceled)

7. A use of fucoxanthin of Chemical Formula 1 or a marine plant extract comprising the same for the preparation of a therapeutic agent for lipid metabolic disorders, wherein the lipid metabolic disorders are selected from the group consisting of diabetes, fatty liver, hyperlipidemia, arteriosclerosis, atherosclerosis, hypertension, cerebral apoplexy and myocardial infarction.

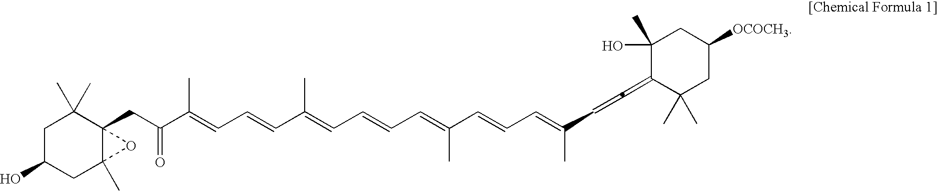

8. A use of fucoxanthin of Chemical Formula 1 or a marine plant extract comprising the same for the preparation of a food composition, wherein the lipid metabolic disorders are selected from the group consisting of diabetes, fatty liver, hyperlipidemia, arteriosclerosis, atherosclerosis, hypertension, cerebral apoplexy and myocardial infarction.

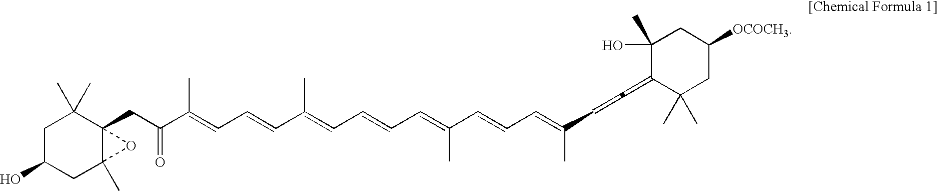

9. A use of fucoxanthin of Chemical Formula 1 or a marine plant extract comprising the same for the preparation of a feed composition, wherein the lipid metabolic disorders are selected from the group consisting of diabetes, fatty liver, hyperlipidemia, arteriosclerosis, atherosclerosis, hypertension, cerebral apoplexy and myocardial infarction.

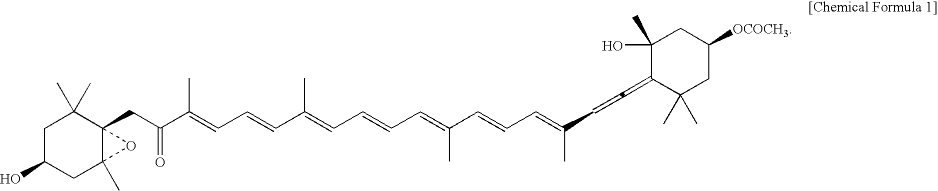

10. A method for preventing or treating lipid metabolic disorders comprising administering fucoxanthin of Chemical Formula 1 or a marine plant extract comprising the same to a subject in need thereof as an effective amount, wherein the lipid metabolic disorders are selected from the group consisting of diabetes, fatty liver, hyperlipidemia, arteriosclerosis, atherosclerosis, hypertension, cerebral apoplexy and myocardial infarction.

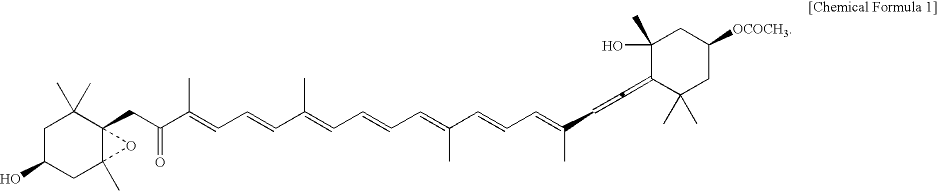

11. The method for preventing or treating lipid metabolic disorders of claim 10, wherein the fucoxanthin or a marine plant extract comprising the same inhibits synthesis of fatty acid or stimulates oxidation of fatty acid.

12. The composition of claim 2, wherein the marine plant are one or more plant which are selected from the group consisting of wakame, dashima, gulfweed and hijiki.

13. The composition of claim 2, wherein the marine plant extract is obtained by extracting marine plants with water, spirit, hexane, ethyl acetate, isopropyl alcohol, acetone or a mixture thereof at 10-50° C. for 1-48 hours.

14. The composition of claim 3, wherein the marine plant are one or more plant which are selected from the group consisting of wakame, dashima, gulfweed and hijiki.

15. The composition of claim 3, wherein the marine plant extract is obtained by extracting marine plants with water, spirit, hexane, ethyl acetate, isopropyl alcohol, acetone or a mixture thereof at 10-50° C. for 1-48 hours.
